(19) United States
(12) Patent Application Publication
(10) Pub. No.: US 2022/0265375 A1
(43) Pub. Date: Aug. 25, 2022

(54) INSTRUMENT FOR A ROBOTIC SURGERY SYSTEM

(71) Applicant: avateramedical GmbH, Jena (DE)

(72) Inventors: Eric MARKWEG, Ilmenau (DE); Thomas BAUR, Rottenburg (DE); Manfred BURKHARD, Dießen (DE)

(21) Appl. No.: 17/670,156

(22) Filed: Feb. 11, 2022

(30) Foreign Application Priority Data
Feb. 25, 2021 (DE) 10 2021 104 516.9

(51) Int. Cl. A61B 34/30; B25J 9/00

(52) U.S. Cl. CPC A61B 34/30; B25J 9/0009; A61B 2034/302

(57) ABSTRACT

An instrument for a robotic surgery system that includes a housing and an outer shaft joined to an effector at a distal end, and to the housing at a proximal end. The connection is rotatory so that the outer shaft can rotate about its longitudinal axis. The rotatory connection takes place by way of a first coupling element that is fixed to the outer shaft. The instrument also includes a first inner shaft arranged within the outer shaft and movably supported relative to it. The relative motion may be either a rotation or translation. The first inner shaft is joined with the effector at a distal end and fixed to a second coupling element at a proximal end. The first coupling element encloses the first inner shaft in a first transition region at the proximal end of the outer shaft, and a first sealing element encloses the first inner shaft.

### PRIORITY CLAIM

The present application claims priority to German Patent Application No. 10 2021 104 516.9, filed on Feb. 25, 2021, which said application is incorporated by reference in its entirety herein.

### FIELD OF THE INVENTION

The invention relates to an instrument for a robotic surgery system that comprises a housing and an outer shaft joined to an effector at a distal end and to the housing at a proximal end. The connection is rotatory so that the outer shaft can rotate about an axis—its longitudinal axis—relative to the housing. The rotatory connection takes place by way of a first coupling element that is fixed with the outer shaft. The instrument further comprises a first inner shaft that is partially arranged within the outer shaft and is movably supported relative to the outer shaft. The relative motion may be either a rotation or a translation. The first inner shaft is joined with the effector at a distal end and fixed to a second coupling element at a proximal end.

### BACKGROUND OF THE INVENTION

Such an instrument employed for minimal-invasive surgery is described, e.g., in the applicant's DE 10 2014 117 408 A1. Where instruments feature several shafts movable relative to each other by translation or rotation, there is always a risk that a patient's body fluids and/or tissue penetrate into the region of the instrument's drive unit and thus cause a contamination of the robotic surgery system.

In prior art, various instruments are known which feature seals for that purpose. US 2012/0209289 A1 describes an instrument in which the drive shaft is enclosed by an O-ring, wherein the O-ring is held form-closed in a recess of a part of the housing so as to be secured against axial shifting. U.S. Pat. No. 7,367,973 B2 describes an instrument wherein two O-rings are arranged within the region of the effector, i.e., at the distal end of the shaft, with one of the O-rings being arranged in a special mount that secures it against axial shifting. US 2017/0290680 A1 also describes the use of a sealing O ring held form-closed within the shaft of the instrument.

US 2016/0193001 A1 describes the use of a silicone ring in the region of the effector between the end cap and the clevis or between the clevis and the end of the effector. The sad document, though, describes the use of such a silicone ring as a disadvantage, and suggests minimizing relevant spacings as a better alternative, which is would facilitate manufacturing and reduce maintenance.

DE 10 2007 030 856 B3 describes a surgical instrument with two shafts—designated there as pistons—movable relative to each other, with a fluid segment between the said shafts. Sealing is provided by a seal fashioned as a flexible, inelastic, fluid-tight membrane and forming an air bellows. One segment is fashioned like a sleeve, while a second segment fills the cross-sectional area of the shaft.

US 2008/0065021 A1 describes an instrument having, at the proximal end, a seal fashioned as a membrane that features a slit and is intended to prevent gas from escaping from the operating site. Die WO 2019/072988 A1 describes an endoscope with a dual seal in axial direction for fluid-tight sealing of the operating channel. The seals are joined to each other and can be manually cleaned or changed without any auxiliaries.

US 2016/0175060 A1 describes an instrument with a reusable shaft and a changeable tip. Sealing between the inner wall of the instrument's tip and the shaft is effected by a sleeve-shaped sealing element of silicone that is movable along a longitudinal axis of the tip.

In US 2010/0168510 A1, the distal end of an instrument is provided with seals, especially in the form of O-rings held form-closed in grooves to prevent leakage of lubricants from the instrument to the operating site.

### SUMMARY OF THE INVENTION

The purpose of the invention is to improve an instrument as described hereinbefore, to the effect that the entry of the patient's body fluids and/or tissue into the region of the drive unit is prevented in the most efficient way, and with the least possible changes of an existing instrument so that the manufacturing process need not be modified; for example, it should be possible to do without the provision of additional grooves or mounts for sealing rings, especially to the effect that existing instrument drive units with instruments coupled to them can continue to be used.

In an instrument of the kind described hereinbefore, this purpose is solved in such a way that the first coupling element encloses the first inner shaft in a first transition region at the proximal end of the outer shaft, and a first sealing element encloses the first inner shaft and seals it against the outer shaft, wherein the sealing element—e.g., a sleeve or a ring—is arranged within the region of the first coupling element. Configured as a flexible, expandable sleeve of sufficient length, the first sealing element may, e.g., enclose partially the first coupling element and partially the first inner shaft: In the assembly process, the sleeve can be pulled onto the first coupling element and the first inner shaft without requiring any added means for fixing the sleeve. The sealing is fluid-tight, i.e., it prevents the passage of fluids.

The seal effect is the better, the longer the sleeve is, measured along the longitudinal axis of the first inner shaft; on the other hand, this may make the motion of the first inner shaft relative to the outer shaft—possibly a rotatory motion or preferably a translatory motion—more difficult: Even though this motion is motor-driven, it may lead to friction losses between the first inner shaft and the first sealing element, with the said friction losses possibly hindering exact positioning of the sleeve and, in the worst case, causing the sleeve to tear. To avoid this, it is expedient to configure the first sealing element with the smallest axial dimension, e.g., as a sealing ring, which, however, requires other ways to ensure that the first sealing element seals the first inner shaft against the outer shaft.

Therein, one takes advantage of the fact that the first coupling element is fixed to the outer shaft, but, at the same time, encloses the first inner shaft in the first transitional region in such a way that the first inner shaft can unimpededly perform the translatory or rotatory motion relative to the outer shaft. This essentially opens up two possible ways, which, in principle, can be combined to enhance the sealing effect: In a first alternative, the first sealing element is fixed to a proximal end of the first coupling element. This fixation can be effected by a substance-to-substance bond, so that no extra holders are required for the first sealing element, which is preferably fashioned as a sealing ring. The first sealing element, then, encloses the first inner shaft at the proximal end of the first coupling element, thus preventing fluid or tissue from entering the inside of the instrument and contaminating the drive unit. In a second alternative, the first sealing element is arranged within the first coupling element in the first transition region, wherein it is axially fixed by the proximal end of the outer shaft and the first coupling element. In this case, one takes advantage of the fact that the radius of the outer shaft is greater than that of the first inner shaft, and thus, the first coupling element must, in the region of the outer shaft, have a grater inside diameter than would be required in the region of the first inner shaft which is also partially enclosed by the first coupling element. Ideally, the region in which the inside diameter of the first coupling element tapers, constitutes an edge and, thus, a stop face for the first sealing element, the said edge preferably extending in a radial direction, with a face normal extending in axial direction. There, the first sealing element can also be fixed by a substance-to-substance bond. Preferably, however, it is also fixed by the proximal end of the outer shaft, which is fixed to the first coupling element, so that it is positively fixed, i.e., clamped between the proximal end of the outer shaft and the stop face, so that one can do without any substance-to-substance bond. This facilitates assembly.

In a particularly preferable embodiment of the first alternative, a first guiding element is arranged between the first coupling element and the second coupling element and, as a rule, joined to the housing. Guided by this guiding element, the first inner shaft is supported with added stability, wherein the first sealing element is arranged between the first coupling element and the first guiding element, by which it is axially fixed. A relative motion between the first coupling element and the first guiding element is not possible, and the distance between the said two elements, measured along the longitudinal axis of the instrument or between the shafts, is dimensioned to the effect that the first sealing element precisely fits between the first coupling element and the first guiding element; axial fixation is form-closed in this case, too, so that one can do without a substance-to-substance bond. The first sealing element can be configured as a sealing ring, e.g., an O ring or an X ring. The first sealing element can contain silicone or be completely made from silicone, with the silicone preferably being biocompatible. The first sealing element is preferably ring-shaped with a rectangular cross-section; at its inner circumference—the bore—and/or at an end face facing the direction of the instrument's segment to be sealed, i.e. here, at the end face facing the first coupling element, the first sealing element features continuously closed sealing blades of tooth-shaped cross-section, which are flexible and elastically deformable, a property that enhances the sealing effect and provides better adaptation to surfaces of higher roughness.

In another embodiment, the instrument further comprises a second inner shaft, which is partially arranged within the first inner shaft, wherein the second inner shaft is movably supported both relatively to the outer shaft and relatively to the first inner shaft—the motion preferably being rotatable, although a translatorily movable bearing is feasible as well. At a distal end, the second inner shaft is also joined to the effector, whereas at a proximal end it is fixed to a third coupling element, wherein the second coupling element encloses the second inner shaft in a second transition region at the proximal end of the first inner shaft, and a second sealing element encloses the second inner shaft and makes it fluid-tight against the first inner shaft, with the second sealing element preferably being configured analogously to the first sealing element, although with a slightly smaller diameter, as a rule.

In a first alternative, the second sealing element is fixed to a proximal end of the second coupling element, for example, by a substance-to-substance bond. Another possibility is to arrange the second sealing element within the second coupling element in the second transition region, where it is then fixed axially by the proximal end of the first inner shaft and the second coupling element, in the way already described above for the first sealing element; these expositions apply analogously to the second sealing element. Both embodiments concerning the arrangement of the second sealing element can, in principle, be combined as well, so as to use two sealing elements, which further improves the sealing against the passage of fluids.

In a preferred embodiment of the instrument in the first alternative for the second sealing element, in which the first inner shaft, relative to the outer shaft, is translatorily shiftable along the axis, and in which one can advantageously do without a substance-to substance fixation of the second sealing element, the instrument comprises a first axial return spring element with a proximal and a distal end, with the said axial return spring element exerting a spring tension on the second coupling element during the latter's translatorily shift from a home position, the spring tension acting in a direction opposite to that shift, with the second sealing element being arranged between, and clamped by, the second coupling element and the first axial return spring element. This clamping is preferably effected in a force-closed and at the same time form-closed manner; an additional fixation is not required. In this way, moreover, a contact pressure on the second sealing element is exerted against the second coupling element, which further improves sealing against the leaking of liquids. A particularly preferable embodiment of this version features a second guiding element that is arranged between the second coupling element and the third coupling element and in which the second inner shaft is guided, wherein the proximal end of the first axial return spring element is placed at the second guiding element or optionally fixed thereon, e.g., by a substance-to-substance bond. Thus, the second guiding element enhances the stability of the instrument and the safety of its use.

In another embodiment of the instrument for effectors with more degrees of freedom, the instrument comprises a third inner shaft, which is partially arranged within the second inner shaft and is supported so as to be movable relative to the outer shaft, the first inner shaft and the second inner shaft. At a distal end, the third inner shaft is joined to the effector, and at a proximal end it is fixed to a fourth coupling element, which encloses the second inner shaft in a third transition region at the proximal end of the second inner shaft. The fourth coupling element is itself tight at the proximal end due to one-piece manufacture, or it is sealed by a closing element fixed by a substance-to-substance bond. Moreover, a third sealing element is fixed at a distal end of the fourth coupling element, encloses the second inner shaft and makes it fluid-tight against the third inner shaft. Here again, the third sealing element may be joined to the distal end of the fourth coupling element by a substance-to-substance bond. The above expositions relating to the special configuration of the first sealing element can analogously be applied to the third sealing element as well and do not need to be repeated here. Due to the substance-to-substance bond between the third sealing element and the fourth coupling element, there is no need here, too, to provide any added fixation means. The exposed arrangement will not impede the making of a reversible connection with an instrument drive unit by a simple click-in.

In an expedient embodiment, the third inner shaft is supported so as to be translatorily shiftable along the axis relative to the outer shaft, relative to the first inner shaft and relative to the second inner shaft. In this case, the instrument with particular preference comprises a second axial return spring element, which with a distal end abuts on the third coupling element or is fixed to it—e.g., by a substance-to-substance bond or in a form-closed manner—and exerts a spring tension on the fourth coupling element during the latter's translatorily shift from a home position, the spring tension acting in a direction opposite to that shift. The third sealing element, then, is arranged between, and clamped by, the distal end of the fourth coupling element and the proximal end of the second axial return spring element, analogously to the second sealing element. Due to the spring tension, clamping is, as a rule, effected at least force-closed and form-closed; the second—and analogously the first—axial return spring element is, as a rule, fitted with a bias. Therefore, no extra fixation, e.g., by a substance-to-substance bond, is required, which facilitates manufacturing.

Expediently, at least one operating element configured, e.g., as a pull rope or pull wire, for the effector is arranged in the third inner shaft.

It is understood that the features mentioned before and those to be explained below are applicable not only in the combinations stated but also in other combinations or as stand-alone features without leaving the scope of the present invention.

### BRIEF DESCRIPTION OF THE DRAWINGS

Below, the invention will be explained in more detail on the basis of exemplary embodiments with reference to the accompanying drawings, which also disclose features essential to the invention. These exemplary embodiments merely serve the purpose of illustration and must not be interpreted as restrictive. For example, a description of an exemplary embodiment featuring a multiplicity of elements or components must not be interpreted in the sense that all these elements or components are needed for an implementation. Rather, other exemplary embodiments may also contain alternative elements and components, fewer elements or components, or additional elements or components. Elements or components of different exemplary embodiments may be combined with each other unless stated otherwise. Modifications and variations described for one of these exemplary embodiments may also be applicable to other exemplary embodiments. To avoid redundancies, similar or mutually corresponding elements in different drawings are designated by the same reference numbers or letters, and explained a single time only. In the drawings:

FIG. 1 is a perspective view of an instrument for a robotic surgery system,

FIG. 2 is a shortened top view of the instrument shown in FIG. 1,

FIG. 3A, shows a section through the tip of the instrument,

FIG. 3B shows a section through the body of the instrument,

FIG. 4 shows a detail of the body shown in FIG. 3B, and

FIG. 5 is a perspective view of a sealing element.

### DETAILED DESCRIPTION

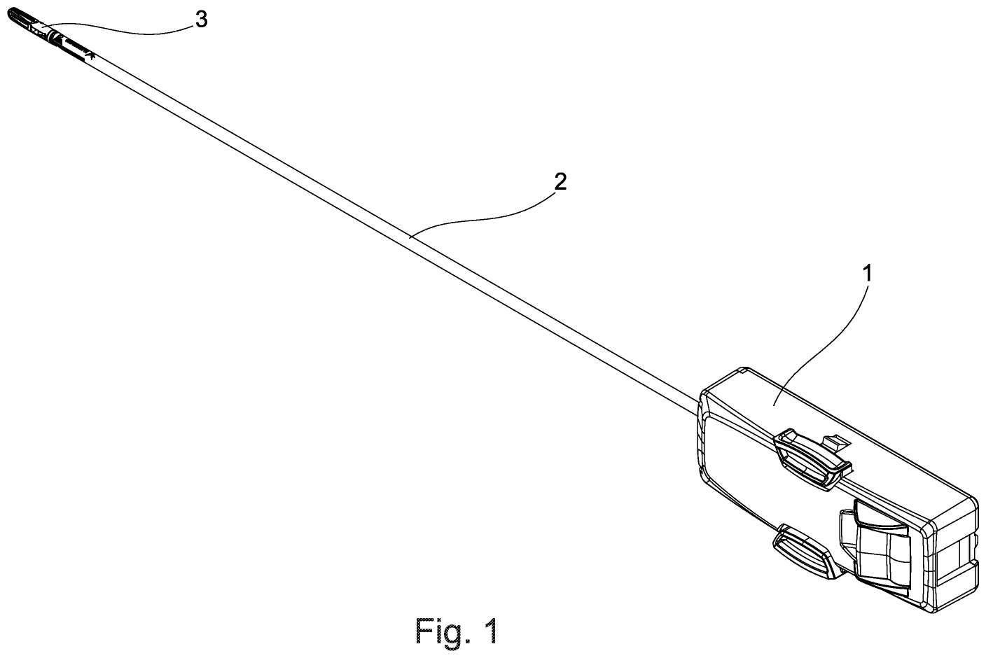

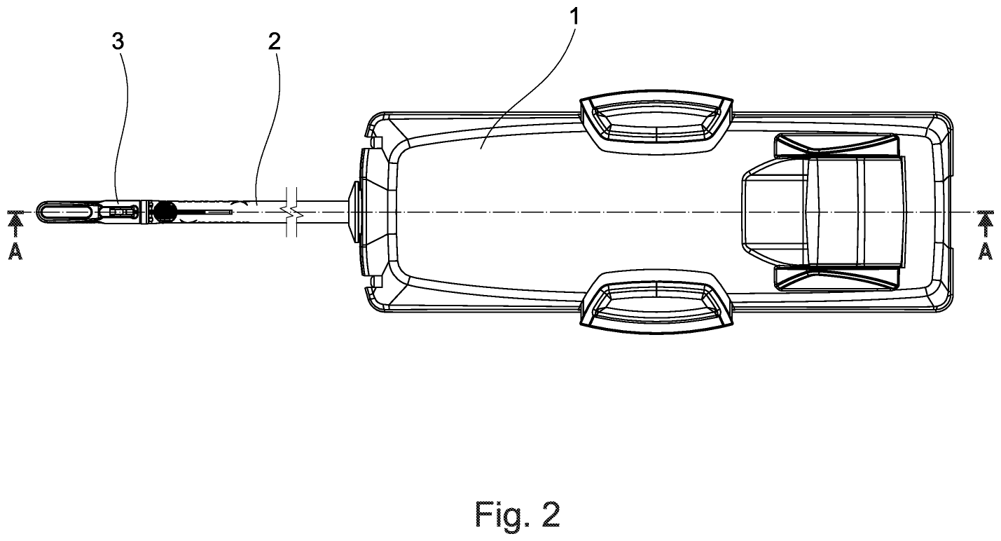

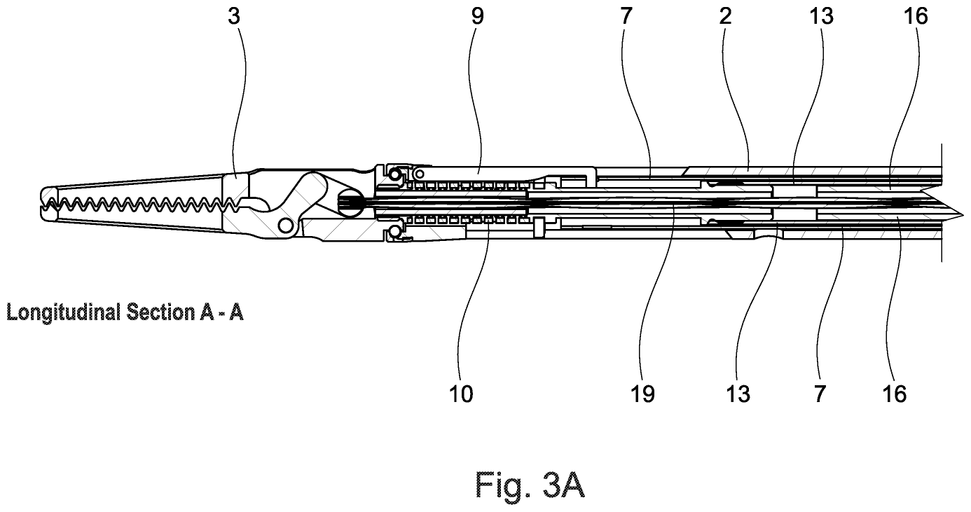

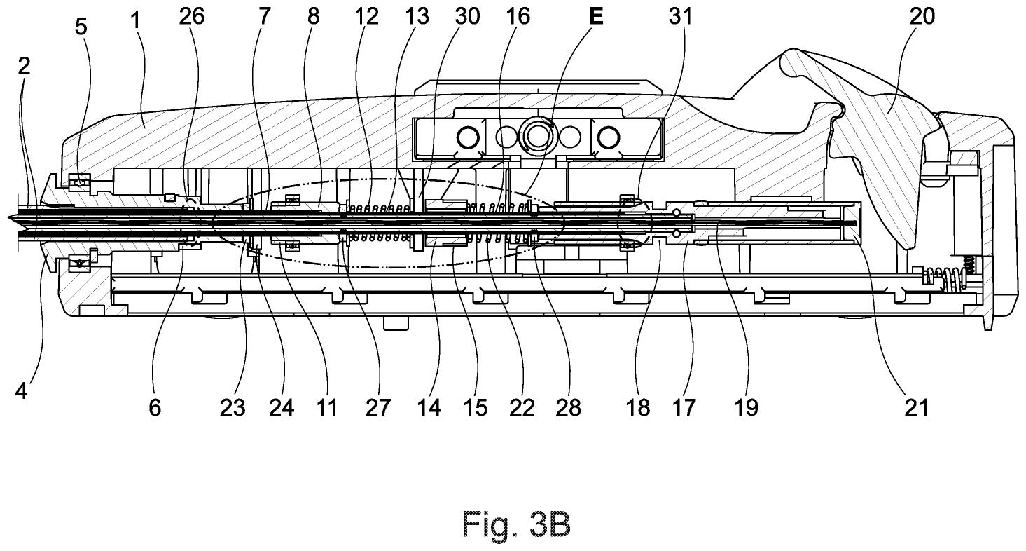

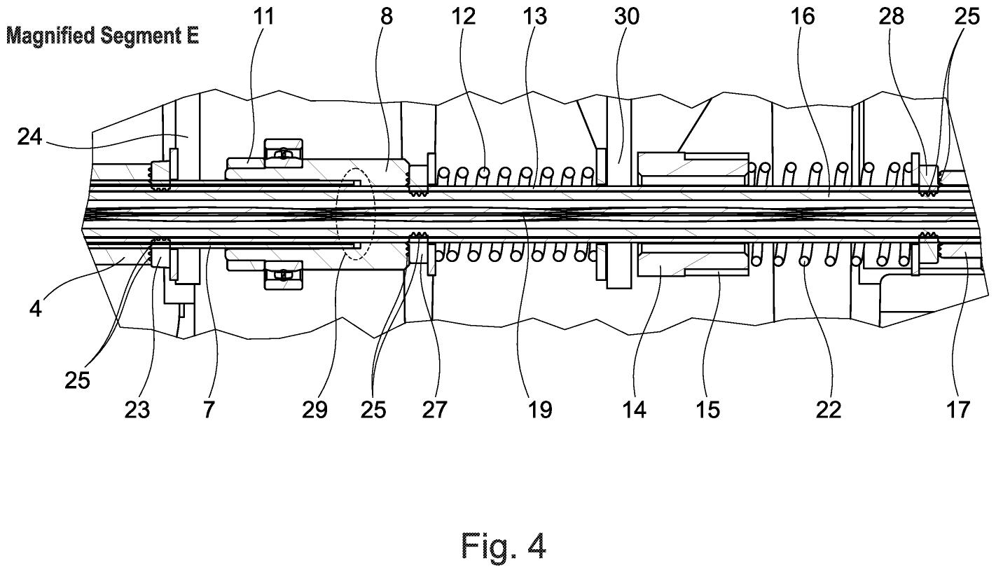

To start with, FIG. 1 shows a perspective view of an instrument for a robotic surgery system. The instrument features a housing 1 shown here from the top. Its bottom side (not visible here) it is reversibly joined to an instrument drive unit by way of a sterile gate. As a rule, the instrument is a one-way article. From the housing 1 there protrudes an instrument shaft, here with an outer shaft 2. The outer shaft 2 is joined to an effector 3 at a distal end, and to the housing 1 at proximal end. Depending on the type of the effector 3, various motions have to be implemented, with the motions being generated by means of the instrument drive unit. If it is a gripping element, for example, the motions that have to be implemented are rotations about the gripping element's own axis, an offset from the longitudinal axis of the instrument shaft, and the opening and closing of the mouth part. This will be explained below in some detail with the help of a longitudinal section A-A through the instrument, as sketched in FIG. 2, which shows the instrument from the top. FIG. 3A shows a segment of the longitudinal section with the effector 3, and FIG. 3B shows a segment of the longitudinal section with the housing 1. FIG. 4 shows a magnified segment marked E in FIG. 3B.

FIG. 3A shows the distal end of the instrument with the effector 3, which here is a gripping element, e.g., a pair of tweezers. However, this is to be understood as an example only; applicable effectors 3 may also be clips, scissors or any other effectors capable of being used in laparoscopic surgery. Effectors with fewer degrees such as, e.g., hooks are eligible as well. The shaft 2 is joined to the housing 1 through a first coupling element 4—shown in FIG. 3B—at the proximal end, wherein the first coupling element 4 is fixed, e.g. bonded, to the outer shaft 2, with the connection of the outer shaft 2 to the housing 1 being effected in such a way as to make the outer shaft 2 rotatable about its longitudinal axis; i.e., this is a rotatory connection, for which purpose the first coupling element 4 is supported in a bearing 5. To implement the rotation, the drive of the instrument driving unit engages with a corresponding first rotatorily driven element that is fixed to, or formed at, the first coupling element 4. In the present example, a first ring gear 6 is press-formed on the first coupling element 4, with the respective drive element engaging with the said ring gear 6. Turning the outer shaft 2 will rotate the instrument about the longitudinal axis of the outer shaft 2.

The instrument further comprises a first inner shaft 7, which is also called a tilting tube shaft. The first inner shaft 7 is partially arranged within the outer shaft 2 and is supported movably relative to the outer shaft 2—here shiftable translatorily along the longitudinal axis. In other embodiments, especially with other effectors, the first inner shaft 7 can be supported so as to be rotatable about the longitudinal axis. The first inner shaft 7, too, is joined to the effector 3 at a distal end. At a proximal end, the first inner shaft 7 is joined to a second coupling element 8. In order to tilt the instrument head, i.e., the effector 3, the first inner shaft 7 can be moved translatorily in the longitudinal direction of the outer shaft 2, which is also called the instrument shaft. With regard to the translatorily motion of the first inner shaft 7, the first coupling element 4 is also used for guiding. For that purpose, the effector 3 is joined to the distal end of the first inner shaft 7 via a tie lever 9. If the first inner shaft 7 is translatorily pulled towards the proximal end, the effector 3 is tilted, and a bending element 10 is bent sideways. A first meshing element 11 at the second coupling element 8, to which the first inner shaft 7 is fixed, serves to transmit the translatory motion from the driving unit to the first inner shaft 7. Return to the initial position, with the effector 3 in axial alignment, is facilitated by a first axial return spring element 12, which exerts a spring tension on the second coupling element 8 when this is translatorily shifted out of an initial position; this spring tension acts in a direction opposite to the said spring tension. Arranged between the first coupling element 4 and the second coupling element 8 is an optional first guiding element 24, in which the first inner shaft 7 is guided in addition.

Arranged partially within the first inner shaft 7 is a second inner shaft 13, which is also supported movably relative to the outer shaft 2 and to the first inner shaft 7. In the example shown here, the second inner shaft 13 is supported rotatably, relative to the outer shaft 2 and to the first inner shaft 7, about the longitudinal axis of the instrument shaft or the outer shaft 2; in this case, the second inner shaft 13 is also called a rotatory tube shaft. In other embodiments of the instrument or of the effector, respectively, the second inner shaft 13 may also be movable translatorily. The second inner shaft 13 is joined to the effector 3 at a distal end, and fixed to a third coupling element 14 at a proximal end. Preferably, that connection is a substance-to-substance bond, but can also be form-closed and/or force-closed. The second coupling element 8 encloses the second inner shaft 13 in a second transition region 29 at the proximal end of the first inner shaft 7, wherein the movability of the second inner shaft 13 relative to the second coupling element 8 or the first inner shaft 7, respectively, will not be impaired. That enclosure should fit as precisely as possible, in order to prevent the entrance of liquid or tissue into the housing 1 as reliably as possible. The same applies to the enclosure of the first inner shaft 7 by the first coupling element 4. To move the second inner shaft 12 rotatorily, the third coupling element 14 features another rotatorily driven element, here, a second ring gear 15 which can be made to rotate about the longitudinal axis by a corresponding drive in the instrument drive unit. The second inner shaft 13 is fixed to the bending element 10 and transmits the rotatory motion to it, those two components being proof against rotation relative to each other. Arranged between the second coupling element 8 and the third coupling element 14 is an optional second guiding element 30, in which the second inner shaft 13 is guided, wherein a proximal end of the first axial return spring element 12 bears against, or is fixed to, the second guiding element 30, in order to give appropriate support to the first axial return spring element 12.

Finally, the instrument comprises a third inner shaft 16, which is partially arranged within the second inner shaft 13 and is movably supported relative to the outer shaft 2, to the first inner shaft 7 and to the second inner shaft 13. In the example shown here, the third inner shaft 16 is supported relative to the other shafts 2, 7 and 13 so as to be translatorily shiftable along the axis, i.e., the longitudinal axis of the outer shaft 2 or the instrument's longitudinal axis, respectively. Depending on a chosen effector, the third inner shaft 16 may deviate by being supported rotatorily. It is also possible for one or several of the inner shafts to 7, 13 or 16 to be left out if necessary.

The third inner shaft 16 is also joined to the effector 3 at a distal end and fixed to a fourth coupling element at a proximal end; the connection is preferably a substance-to-substance bond but may also be effected otherwise. The fourth coupling element 17 encloses the second inner shaft 13 in a third transition region 31 at the proximal end of the second inner shaft 13. The translatory motion generated by the instrument drive unit is transmitted to the fourth coupling element 17 via a second meshing element 18 fashioned at it. Here, the second meshing element 18 is configured as a peripheral outer groove; its radially extending bounding faces serve as stop faces for the drive. Situated within the third inner shaft 16—also called a gripping tube shaft—there is an actuating element for the effector 3, here a pull wire 19, with which a gripping or cutting motion of the effector 3 can be released. Via the instrument drive unit, the pull wire 19 is shifted in the distal direction to open the legs of the gripping element of the effector 3. By means of a lever element 20, the gripping element can be opened manually, e.g., in case of a failure of the system or in case of other defects that prevent opening by the instrument drive by means of the control. For that purpose, the lever element is moved clockwise about a rotary axis extending normal to the drawing plane, and hits a closing element 21, which is joined to the fourth coupling element 17 by a substance-to-substance bond and seals it fluid-tight. Here, the pull wire 19 is fixed inside the closing element 21, but it may also be fixed directly to the fourth coupling element 17. To close the gripping element, the pull wire 19 is moved in the opposite (proximal) direction. To ensure firm and secure gripping, a second axial return spring element 22 exerts an additional thrust force on the pull wire 19 and the fourth coupling element 17; during opening, the second axial return spring element 22 is compressed. In the present example, the second axial return spring element 22 is fixed to the third coupling element 14, which is firmly positioned axially.

Herein, the outer shaft 2, the first inner shaft 7, the second inner shaft 13 and the third inner shaft 16 are arranged in a coaxial manner and coupled to each other so as to allow a rotary motion to be transferred. If, then, the outer shaft 2 is rotated, the first inner shaft 7, the second inner shaft 13 and the third inner shaft 16 rotate equally so that their positions relative to each other remain the same. If, however, only the second inner shaft 13 is rotated, only the third inner shaft 16 will rotate together with it.

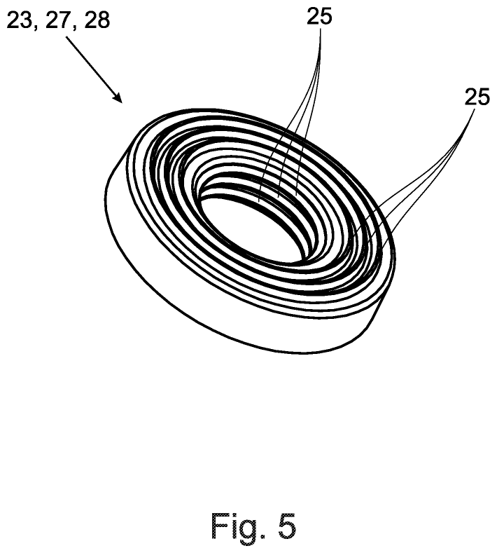

With precisely fitting dimensions of the coupling elements 4, 8, 14, 17 in the transition regions, where they enclose the respective next inner shaft, the penetration of body fluids and tissues into the housing 1 can essentially be suppressed, though, but not excluded completely. To efficiently avoid the penetration of body fluids into the housing 1, the instrument comprises several sealing elements. A first sealing element 23 encloses the first inner shaft 7 and seals it against the outer shaft 2. In the present example, the first inner shaft 7 is guided in the first guiding element 24, which is arranged between the first coupling element 4 and the second coupling element 8. The first sealing element 23 is arranged between the first coupling element 4 and the first guiding element 24. It is axially fixed by these two elements, because the first guiding element 24 is fixed to the housing 1, and the first coupling element 4 is axially fixed, too. This positioning of the first sealing element 23 makes it possible to easily retrofit existing systems and to modify the manufacturing process, without requiring all manufacturing tools, e.g., injection molds, to be modified. The sealing element 23 is of a ring-shaped configuration. In the example shown in FIG. 4, the first sealing element 23 has a rectangular cross-section. FIG. 5 shows the details of the first sealing element 23 in a perspective view. Peripherally closed, elastic barriers or sealing blades 25 of tooth-shape cross-section are configured on the sides that, during operation, point towards the coupling elements and to the respective shaft, i.e., in the direction of the parts to be sealed; these sealing blades 25 form a series of liquid barriers arranged one behind the other, thus further enhancing the sealing effect.

Alternatively, one can do without the first guiding element 24; instead, the first sealing element 23 can be directly fixed to a proximal end of the first coupling element 4, for example, by a substance-to-substance bond. In another alternative embodiment, the first sealing element 23 can be arranged within the first coupling element 4 in a first transition region 26. There it is axially fixed by the proximal end of the outer shaft 2 and by the first coupling element 4. In none of these cases would it be necessary to substantially modify the tools and the process for manufacturing the instrument, apart from the need to integrate the first sealing element 23 in an additional step.

The first sealing element 23 seals the first inner shaft 7 against the outer shaft 2. For sealing the second inner shaft 13 against the first inner shaft 7, a second sealing element 27 is provided, which encloses the second inner shaft. For sealing the second inner shaft 13 against the den third inner shaft 16, a third sealing element 28 is provided, which also encloses the second inner shaft 13, because it is arranged at the distal end of the fourth coupling element 17. All three sealing elements 23, 27 and 28 can be configured similarly. An eligible material may be silicone, especially biocompatible silicone.

Here, the second sealing element 27 is arranged, and clamped, between the first axial return spring element 12 and the second coupling element 8; additional fixation to the first axial return spring element 12 and/or to the second coupling element 8, while possible, is not required. In case of a substance-to-substance bond, care should be taken to use an adhesive that does not lead to a curing of the material of the sealing elements, as this could impair the sealing effect. Preferably, therefore, where a substance-to-substance fixation is intended, the sealing elements are fixed to the sides on which no liquid is expected to penetrate. Particularly eligible in case of the second sealing element 27 would therefore be fixation to the first axial return spring element 12, whereas the third sealing element 28 should rather be fixed to the second axial return spring element 22. Alternatively, the second sealing element 27 can be fixed to a proximal end of the second coupling element 8 only. In yet another alternative embodiment, the second sealing element 27 can be arranged within the second coupling element 8 in the second transition region 29, where it is axially fixed by the proximal end of the first inner shaft 7 and the second coupling element 8.

Due to the use of sealing elements, the instrument described above is better sealed than known instruments of similar design. The arrangement of the sealing elements can be adopted by existing instrument configurations without major changes. Thanks to the use of several sealing elements and to their distribution along the longitudinal axis at the transition points or in the transition regions between the shafts, all points with probabilities for liquid to penetrate into the housing can efficiently be sealed. One can do without any additional fixation of the sealing elements, as these are already fixed in their axial positions by existing return spring elements, coupling elements and/or guiding elements. Both in rotatory and translatory motion, the shafts can slide below the sealing elements, so that these will not be shifted unintentionally.

### LIST OF REFERENCE NUMBERS

1 housing
2 outer shaft
3 effector
4 first coupling element
5 bearing
6 first ring gear
7 first inner shaft
8 second coupling element
9 tie lever
10 bending element
11 first meshing element
12 first axial return spring element
13 second inner shaft
14 third coupling element
15 second ring gear
16 third inner shaft
17 fourth coupling element
18 second meshing element
19 pull wire
20 lever element
21 closing element
22 second axial return spring element
23 first sealing element
24 first guiding element
25 sealing blade
26 first transition region
27 second sealing element
28 third sealing element
29 second transition region
30 second guiding element
31 third transition region

What is claimed is:

1. An instrument for a robotic surgery system, comprising:
a housing,
an outer shaft which is, at a distal end, joined to an effector and, at a proximal end, rotatorily joined to the housing via a first coupling element fixed to the outer shaft so that the outer shaft can rotate about an axis,
a first inner shaft which is partially arranged within the outer shaft and supported so as to be movable relative to the outer shaft, and which is joined to the effector at a distal end and fixed to a second coupling element at a proximal end, and
wherein the first coupling element encloses the first inner shaft in a first transition region at a proximal end of the outer shaft, and
wherein a first sealing element encloses the first inner shaft and is fixed to a proximal end of the first coupling element or is arranged within the first coupling element in the first transition region and axially fixed by the proximal end of the outer shaft and the first coupling element, thusly sealing the first inner shaft against the outer shaft.

2. The instrument as claimed in claim 1, wherein the first inner shaft is supported so as to be translatorily shiftable along the axis relative to the outer shaft.

3. The instrument as claimed in claim 1, wherein the first inner shaft is fixed to a proximal end of the first coupling element, and a first guiding element is arranged between the first coupling element and the second coupling element, providing guidance to the first inner shaft, and the first sealing element is arranged between, and axially fixed by, the first coupling element and the first guiding element.

4. The instrument as claimed in claim 1, further comprising:
a second inner shaft that is partially arranged within the first inner shaft and supported movably relative to the shaft and to the first inner shaft, and that is joined to the effector at a distal end and fixed to a third coupling element at a proximal end, and
wherein the second coupling element encloses the second inner shaft at the proximal end of the first inner shaft in a second transition region, and
wherein a second sealing element encloses the second inner shaft, and is fixed to a proximal end of the second coupling element or is arranged within the second coupling element in the second transition region and axially fixed there by the proximal end of the first inner shaft and the second coupling element, thusly sealing the second inner shaft against the first inner shaft.

5. The instrument as claimed in claim 4, wherein the second inner shaft is supported so as to be rotatable about the axis relative to the outer shaft and to the first inner shaft.

6. The instrument as claimed in claim 4, wherein the second sealing element is fixed to a proximal end of the second coupling element, and the first inner shaft is supported so as to be translatorily shiftable along the axis relative to the outer shaft, comprising a first axial return spring element that has a proximal and a distal end and exerts a spring force onto the second coupling element during the second coupling element's translatory shift out of an initial position, the said spring force acting in a direction opposite to the said shift, wherein the second sealing element is arranged between, and clamped by, the second coupling element and the first axial return spring element.

7. The instrument as claimed in claim 6, further comprising a second guiding element which is arranged between the second coupling element and the third coupling element and in which the second inner shaft is guided, and wherein the proximal end of the first axial return spring element bears against, or is fixed to, the second guiding element.

8. The instrument as claimed in claim 4, further comprising:
a third inner shaft which is partially arranged within the second inner shaft and is supported movably relative to the outer shaft, to the first inner shaft and to the second inner shaft, and which is joined to the effector at a distal end and fixed to a fourth coupling element at a proximal end, and
wherein the fourth coupling element encloses the second inner shaft in a third transition region at the proximal end of the second inner shaft, and
wherein a third sealing element is fixed to a distal end of the fourth coupling element, encloses the second inner shaft, and seals it against the third inner shaft.

9. The instrument as claimed in claim 8, wherein the third inner shaft is supported so as to be translatorily shiftable along the axis relative to the outer shaft, to the first inner shaft and to the second inner shaft.

10. The instrument as claimed in claim 8, further comprising a second axial return spring element, a distal end of which bears against, or is fixed to, the third coupling element and which exerts a spring force onto the fourth coupling element during the fourth coupling element's translatory shift out of an initial position, the said spring force acting in a direction opposite to the said shift, wherein the third sealing element is arranged between, and clamped by, the distal end of the fourth coupling element and the proximal end of the second axial return spring element.

11. The instrument as claimed in claim 8, wherein, in the third inner shaft, at least one actuation element for the effector is arranged, which is configured as a pull rope or pull wire.

12. The instrument as claimed in claim 1, wherein the first sealing element and the second sealing element are configured as sealing rings.

13. The instrument as claimed in claim 12, wherein the sealing rings have a rectangular cross-section and, at an inner periphery and/or at least on one end face, are configured as peripherally closed lamellae of a tooth-shaped cross-section.

14. The instrument as claimed in claim 1, wherein the first sealing element and the second sealing element are made of, or contain, silicone.
